(19) United States
(12) Patent Application Publication
(10) Pub. No.: US 2025/0022586 A1
(43) Pub. Date: Jan. 16, 2025

(54) MEDICAL PROCEDURE CASE SCHEDULING

(71) Applicant: Medtronic Vascular, Inc., Santa Rosa, CA (US)

(72) Inventors: Max H. Billard, Petaluma, CA (US); Paul J. Coates, Corte Madera, CA (US)

(21) Appl. No.: 18/758,173

(22) Filed: Jun. 28, 2024

Related U.S. Application Data
(60) Provisional application No. 63/512,799, filed on Jul. 10, 2023.

(51) Int. Cl. G16H 40/20

(52) U.S. Cl. CPC G16H 40/20

(57) ABSTRACT

Example medical systems and techniques are disclosed. An example medical system includes processing circuitry coupled to memory. The processing circuitry is configured to determine at least one of a predicted duration or a predicted end time of a medical procedure. The processing circuitry is configured to monitor data associated with the current medical procedure during the current medical procedure and determine that an event occurs in the current medical procedure at a first time based on the data associated with the current medical procedure. The processing circuitry is configured to determine at least one of a revised predicted duration or a revised predicted end time of the current medical procedure based on the event occurring at the first time and output a scheduling indication for a subsequent medical procedure based on the at least one of the revised predicted duration or the revised predicted end time.

### RELATED APPLICATIONS

This application claims the benefit of U.S. Provisional Application Ser. No. 63/512,799, filed Jul. 10, 2023, the entire contents of each of which are incorporated herein by reference.

### TECHNICAL FIELD

This disclosure relates to scheduling of medical procedures.

### BACKGROUND

Medical procedures may include diagnostic and interventional medical procedures. Sometimes such medical procedures may take more time or less time than expected. In some examples, a medical procedure that begins as diagnostic medical procedure may become an interventional medical procedure, such as when a clinician discovers a medical issue during a diagnostic procedure that requires immediate medical attention. Such an event may cause the amount of time for which the medical procedure was scheduled or estimated to exceed the allotted time, which may impact other scheduled medical procedures.

### SUMMARY

Medical procedures performed in an operating room or a Catheterization (Cath) lab are typically scheduled according to a certain predetermined cadence, sometimes including buffers in between procedures, to minimize wasted time by clinicians, such as doctors and key staff. For example, each medical procedure may be allotted a duration of time during which hospital staff, equipment, rooms and/or the like, may be dedicated to the medical procedure. These predetermined cadences and buffers may be carefully considered, but ultimately are based on limited data. Patients often wait significantly longer for their medical procedure to start than they were led to expect. In some instances, a patient's medical procedure may need to be rescheduled to another day or another facility due to unforeseen or unaccounted for delays in medical procedures prior to the patient's medical procedure. It is fairly common for an upcoming medical procedure to be rescheduled due to prior medical procedures taking significantly longer than expected.

Patients waiting for a medical procedure are likely already uncomfortable and/or in a state of reduced health. Requiring a patient to remain in a waiting room for a longer period of time, to have to return on a second day, and/or to travel to another facility, may exasperate patient discomfort, increase exposure of the patient to other patients (which may increase a risk of communicable disease, fungus, infection, etc.), increase patient stress, patient expense, patient driver lost time, and/or patient inconvenience.

As such, it may be desirable to improve medical procedure scheduling so as to reschedule medical procedures which are likely to be impacted based on events occurring during a current medical procedure.

It also may be an inefficient use of medical facility resources to schedule buffer times between procedures, if such buffer times are not necessary. For example, if a buffer time is scheduled after a medical procedure's expected end time and the medical procedure ends on time or early, medical facility resources, such as the room, medical equipment, and/or clinicians, may be waiting unused until after the buffer period expires.

In general, this disclosure is directed to various techniques and medical systems for revising schedules for future medical procedures based on events occurring during a current medical procedure. For example, a medical system may determine that an event occurs during the current medical procedure. The event may be indicative of the end of a phase of the medical procedure, the beginning of a phase of the medical procedure, a patient reaction or the like, which may be indicative of a change in an expected or predicted duration or end time of the current medical event. If the predicted duration or end time of the current medical procedure is revised, the medical system may output a scheduling indication and/or reschedule a future medical procedure.

In one example, the disclosure describes a medical system comprising: memory configured to store at least one of a predicted duration or predicted end time of a current medical procedure; and processing circuitry communicatively coupled to the memory, the processing circuitry being configured to: determine at least one of the predicted duration or the predicted end time; monitor data associated with the current medical procedure during the current medical procedure; determine that an event occurs in the current medical procedure at a first time based on the data associated with the current medical procedure; determine at least one of a revised predicted duration or a revised predicted end time of the current medical procedure based on the event occurring at the first time; and output a scheduling indication for a subsequent medical procedure based on the at least one of the revised predicted duration or the revised predicted end time.

In another example, the disclosure describes a method comprising: determining, by processing circuitry, at least one of a predicted duration or a predicted end time of a current medical procedure; monitoring, by the processing circuitry, data associated with the current medical procedure during the current medical procedure; determining, by the processing circuitry, that an event occurs in the current medical procedure at a first time based on the data associated with the current medical procedure; determining, by the processing circuitry, at least one of a revised predicted duration or a revised predicted end time of the current medical procedure based on the event occurring at the first time; and outputting, by the processing circuitry, a scheduling indication for a subsequent medical procedure based on the at least one of the revised predicted duration or the revised predicted end time.

In yet another example, the disclosure describes a non-transitory computer readable medium comprising instructions, which when executed, cause processing circuitry to: determine at least one of a predicted duration or a predicted end time of a current medical procedure; monitor data associated with the current medical procedure during the current medical procedure; determine that an event occurs in the current medical procedure at a first time based on the data associated with the current medical procedure; determine at least one of a revised predicted duration or a revised predicted end time of the current medical procedure based on the event occurring at the first time; and output a scheduling indication for a subsequent medical procedure based on the at least one of the revised predicted duration or the revised predicted end time.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for rescheduling a future medical procedure according to one or more aspects of this disclosure.

FIG. 2 is a schematic view of one example of a computing system of the system of FIG. 1.

FIG. 3 is a tabular diagram illustrating example inputs that may be used to determine a duration or an end time of a medical procedure according to one or more aspects of this disclosure.

FIG. 4 is a flow diagram illustrating example medical procedure rescheduling techniques according to one or more aspects of this disclosure.

FIG. 5 is a conceptual diagram illustrating an example machine learning model according to one or more aspects of this disclosure.

FIG. 6 is a conceptual diagram illustrating an example training process for a machine learning model according to one or more aspects of this disclosure.

### DETAILED DESCRIPTION

As mentioned above, medical procedures performed in an operating room or a Cath lab are typically scheduled following a certain predetermined cadence, sometimes including buffers in between procedures, to minimize wasted time by clinicians, such as doctors and key staff. However, some medical procedures may take significantly longer than scheduled, even with a buffer period, due to unforeseen complications or other issues which may arise or be discovered during the medical procedure. Such medical procedures may impact scheduling of subsequent medical procedures.

Rescheduling a patient of a subsequent medical procedure may cause additional stress, discomfort, and/or inconvenience for the patient and may increase a risk that the patient be exposed to communicable disease, fungus, infection, as the patient may unnecessarily spend more time in a medical facility waiting room due to the rescheduling. In some cases, a medical procedure may be completed early or before expiration of the buffer period, thereby leaving medical facility resources unused until the start of the next scheduled medical procedure.

According to the techniques of this disclosure, a medical system may predict or estimate a predicted duration or end time of a medical procedure. The medical system may monitor and log medical procedure time and significant events (e.g., milestones) within the medical procedure. The medical system may use such information to assist with subsequent and/or current medical procedure scheduling, to provide live (e.g., real-time) medical procedure duration projections, and assess the impact on upcoming medical procedures, for example, subsequent medical procedures scheduled for that same day that may be affected by the extension or contraction of a duration of a current medical procedure. It should be noted that the predicted duration or end time may be represented in any number of formats, such as a median or mean, a confidence interval, a historical range, or the like.

Additionally, facility or individual medical procedure techniques (e.g., surgical techniques) are currently changed in an ad hoc manner. For example, one clinician may notice that different techniques may reduce medical procedure time, or periodic audits may flag a particular clinician as being associated with relatively long (or speedy) medical procedure times, but additional information would need to be gathered to determine why the particular clinician is involved in longer or shorter (in time) medical procedures. Improvements in medical procedure techniques inspired by other facilities are even more unusual with many of such improvements coming from yearly conventions, journal articles, or new staff. The techniques of this disclosure may identify medical techniques which are more efficient than other medical techniques and, as such, facilitate proliferation of such techniques.

The techniques of this disclosure may not only provide scheduling staff with a better initial estimate of how long medical procedures typically take, but may provide scheduling staff with real-time notice of extending medical procedure times. This real-time notice may be used to better manage rescheduling of upcoming medical procedures, patient expectations, and patient requirements or burdens, thus improving hospital resource allocation and patient experience. Additionally, the techniques of this disclosure may provide a medical facility with information that could help them better match clinicians, such as staff, physicians, and technicians, with a particular upcoming medical procedure and/or with each other, to improve medical procedure time. The techniques of this disclosure may also provide evidence of successful best practices or technology other facilities are implementing.

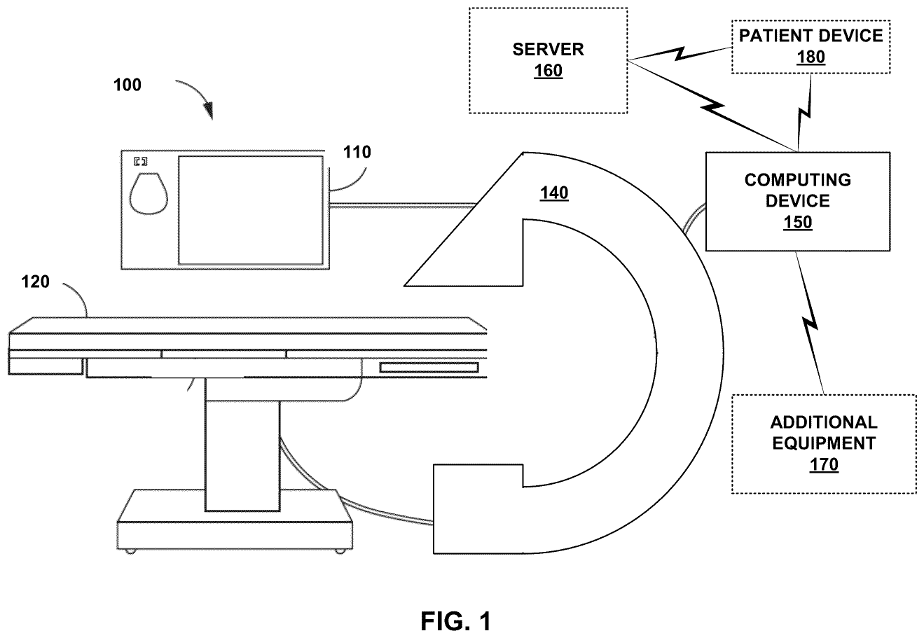

FIG. 1 is a schematic perspective view of one example of a system for determining a recommended treatment strategy according to one or more aspects of this disclosure. System 100 includes a display device 110, a table 120, an imager 140, and a computing device 150. In some examples, system 100 includes additional equipment 170, such as one or more cameras which may capture video image data of occurrences in the environment surrounding system 100, one or more microphones which may capture audio data of occurrences in the environment surrounding system 100, a procedural device (e.g., a renal artery denervation (RDN) device, a laparoscopic tool, a surgical tool, etc.) which may be used to treat a patient, an anesthesia delivery device, an FFR device, a barcode scanner which may be configured to scan a barcode of a medical instrument or device indicative of the identity of the medical instrument or device, and/or other medical instruments or medical devices. System 100 may be an example of a system for use in a medical facility, such as in an operating room or a Cath lab. In some examples, system 100 may include other devices, not shown for simplicity purposes. In some examples, system 100 may also include server 160, which may be co-located with the other devices of system 100 or may be located elsewhere. System 100 may be used during a medical procedure, such as a diagnostic and/or an interventional medical procedure to treat a medical condition of a patient. System 100 may determine a predicted duration and/or end time of a medical procedure. During such a medical procedure, system 100 may monitor data associated with the medical procedure and revise a predicted duration and/or end time of the medical procedure based on data collected by system 100, according to the techniques of this disclosure.

For example, computing device 150 may be coupled to a plurality of common pieces of equipment that may provide data to computing device 150. Such equipment may include imager 140, which may include a C-Arm imager, or other imager, and additional equipment 170, which may include equipment monitoring a patient's vitals, one or more cameras, one or more microphones, or the like. In some examples, additional equipment 170 may include specialty equipment, such as an RDN generator, an IVUS imaging device (which may be an example of imager 140), specialty FFR estimating equipment or reports, as well as relevant anesthesia equipment, or the like. In some examples, the connections between imager 140, additional equipment 170 and computing device 150 may include physical audio/visual (A/V) and/or data ports.

System 100 may include one or more machine learning models. A machine learning model may be trained to determine a predicted duration and/or end time of a medical procedure. Additionally, or alternatively, the machine learning model may be trained to, based on data associated with the medical procedure, such as image data, audio data, video data, and/or other data, determine a revised duration and/or end time for the medical procedure. For example, the machine learning model may be trained to determine an event, such as the end of a phase of the medical procedure, the beginning of a phase of the medical procedure, the use of an unexpected medical instrument, or the like, based on data associated with the medical procedure.

System 100 may use the revised duration and/or end time to automatically reschedule other subsequent medical procedures that may be impacted by the revised duration or end time of the current medical procedure or to alert medical facility staff and/or an impacted patient of the likelihood that the subsequent medical procedure may need to be rescheduled.

In some examples, system 100 may communicate with a computing device, patient device 180, associated with a patient of a subsequent medical procedure, such as to notify the patient of the subsequent medical procedure of a change in scheduling of their medical procedure. Patient device 180 may include a mobile device, such as a smartphone or other cellular phone, a tablet computer, a laptop computer, a desktop computer, or the like. For example, computing device 150 and/or server 160 may communicate with patient device 180 via automated phone call, short message service (SMS) text message, email, web portal notification, or the like.

Computing device 150 may include, for example, an off-the-shelf device such as a laptop computer, desktop computer, tablet computer, smart phone, or other similar device or may include a specific purpose device. Computing device 150 may perform various control functions with respect to imager 140. In some examples, computing device 150 may include a guidance workstation. Computing device 150 may control the operation of imager 140 and receive the output of imager 140.

Display device 110 may be configured to output instructions, images, and messages relating to the medical procedure. Table 120 may be, for example, an operating table or other table suitable for use during a medical procedure.

In the example of FIG. 1, imager 140, such as a fluoroscopy imager, an angiography imager, or other imaging device, may be used to image relevant portions of the patient's anatomy during the medical procedure to visualize the anatomy, characteristics and locations of lesions or other issues inside the patient's body through the generation of imaging data. While described primarily as a fluoroscopy imager, imager 140 may be any type of imaging device, such as an angiography device, an intravascular ultrasound (IVUS) device, an optical coherence tomography (OCT)-fractional flow reserve (FFR) device, a computed tomography (CT) device, a magnetic resonance imaging (MRI) device, a positron emission tomography (PET) device, an ultrasound device, or the like. In some examples, imager 140 may represent more than one imaging device, such as a plurality of any of the aforementioned devices.

Imager 140 may image a region of interest in the patient's body. The particular region of interest may be dependent on anatomy, the medical procedure, patient symptoms, and/or the like. For example, when performing a cardiac medical procedure, a portion of the vasculature and/or the heart may be within the region of interest.

Computing device 150 may be communicatively coupled to imager 140, display device 110 and/or server 160, for example, by wired, optical, or wireless communications. Server 160 may be a hospital server which may or may not be located in an emergency room or Cath lab of a hospital, a cloud-based server, or the like. Server 160 may be configured to store patient imaging data, electronic healthcare or medical records, or the like.

Any of, or any combination of, computing device 150, imager 140, and/or server 160 may include one or more machine learning model(s). For example, computing device 150, imager 140, and/or server 160 may obtain image data of the cardiac anatomy of the patient. Computing device 150, imager 140, and/or server 160 may, based on data associated with a medical procedure determine that an event occurs during the medical procedure that may impact a predicted duration or end time of the medical procedure. In some examples, computing device 150, imager 140, and/or server 160 may execute a machine learning model to determine a predicted duration and/or end time for a medical procedure. For example, computing device 150, imager 140, and/or server 160 in executing the machine learning model may determine confidence intervals which may be used to determine the predicted duration and/or end time for the medical procedure. In some examples, computing device 150, imager 140, and/or server 160 may execute a machine learning model to determine the event occurs and/or to determine a revised duration or end time for the medical procedure. In some examples, computing device 150, imager 140, and/or server 160 may output a scheduling revision for a subsequent medical procedure based on the revised duration and/or end time of the current medical procedure.

By outputting a scheduling revision for the subsequent medical procedure, system 100 may improve the efficiency of medical facility resource usage and/or reduce patient stress, discomfort, and/or inconvenience due to unexpected, “last minute” rescheduling of a subsequent medical procedure.

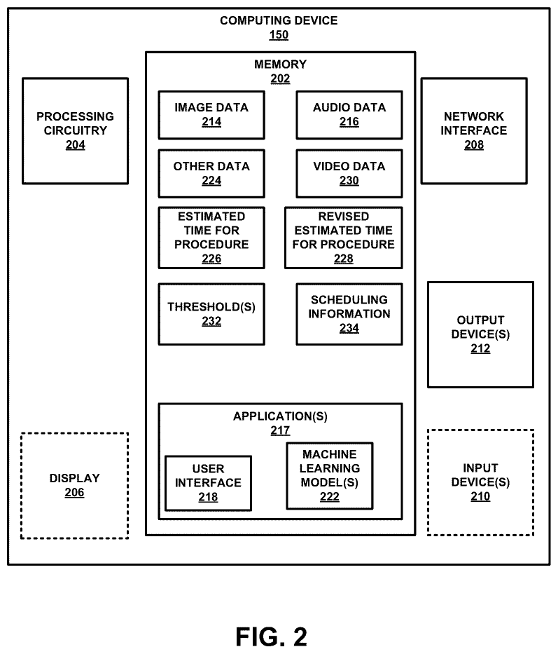

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with imager 140. As shown in FIG. 2, computing device 150 may represent multiple instances of computing devices, each of which may be associated with imager 140. Computing device 150 may include, for example, a memory 202, processing circuitry 204, a display 206, a network interface 208, input device(s) 210, and/or output device(s) 212, each of which may represent any of multiple instances of such a device within the computing system, for case of description.

While processing circuitry 204 appears in computing device 150 in FIG. 2, in some examples, features attributed to processing circuitry 204 may be performed by processing circuitry of any of computing device 150, imager 140, or server 160, or combinations thereof. In some examples, one or more processors associated with processing circuitry 204 in computing system may be distributed and shared across any combination of computing device 150, imager 140, and server 160. Computing device 150 may be used to perform any of the techniques described in this disclosure, and may form all or part of devices or systems configured to perform such techniques, alone or in conjunction with other components, such as components of computing device 150, imager 140, server 160, or a system including any or all of such systems.

Memory 202 of computing device 150 includes any non-transitory computer-readable storage media for storing data or software that is executable by processing circuitry 204 and that controls the operation of computing device 150 and/or imager 140, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store image data 214, audio data 216, other data 224, video data 230, estimated time for procedure 226, revised estimated time for procedure 228, threshold(s) 232, and/or scheduling information 234. Image data 214 may be captured by imager 140 (FIG. 1) during a medical procedure of a patient. Processing circuitry 204 may obtain imaging data 214 from imager 140 and store image data 214 in memory 202. In some examples, image data 214 may include image data of the patient captured during a previous or prior medical procedure as well as imaged data of the patient captured during a current medical procedure. Processing circuitry 204 may execute user interface 218 so as to cause display 206 (and/or display device 110 of FIG. 1) to present user interface 218 to one or more clinicians performing the medical procedure. For example, image data 214 may be generated by imager 140 of anatomy of the patient and obtained by computing device 150 via network interface 208 which may be communicatively coupled to imager 140. In some examples, image data 214 may include image data indicative of medical devices and/or medical instruments being implanted or used during a medical procedure, such as a stent, sternal ties, balloons, guidewires, or the like.

Memory 202 may store audio data 216. For example, additional equipment 170 (FIG. 1) may include one or more microphones which may capture audio data. Processing circuitry 204 may obtain the audio data from additional equipment 170 via network interface 208 and store the obtained audio data in audio data 216. Audio data 216 may be indicative of events occurring during a medical procedure. For example, a clinician may verbally ask a nurse for a particular medical instrument which may be indicative of that medical instrument being deployed during the medical procedure.

Memory 202 may store other data 224. For example, a clinician may input via input device(s) 210 other information relevant to the medical procedure, such as information identifying the clinicians performing the medical procedure, roles for each such clinician, a type of procedure, equipment to be used during the procedure, or the like. Other data 224 may also include data from additional equipment 170.

Memory 202 may store video data 230. For example, additional equipment 170 (FIG. 1) may include one or more cameras which may capture video data. Processing circuitry 204 may obtain the video data from additional equipment 170 via network interface 208 and store the obtained video data in video data 230. Video data 230 may be indicative of events occurring during a medical procedure. For example, a clinician may pick up or move a particular medical instrument which may be indicative of that medical instrument being deployed during the medical procedure.

Memory 202 may store estimated time for procedure 226. Estimated time for procedure 226 may include an estimated duration of the medical procedure and/or an estimated end time for the medical procedure. In some examples, estimated time for procedure 226 may be generated by processing circuitry 204 executing machine learning model(s) 222. In other examples, estimated time for procedure 226 may be entered by a clinician or scheduling staff via input device(s) 210 or on another computing device via network interface 208.

Memory 202 may store revised estimated time for procedure 228. Revised estimated time for procedure 228 may include a revised estimated duration of the medical procedure and/or a revised estimated end time for the medical procedure. For example, processing circuitry 204 executing machine learning model(s) 222 may determine an event occurs during a current medical procedure. Processing circuitry 204 executing machine learning model(s) 222 may determine revised estimated time for procedure 228 based on the event occurring.

Memory 202 may store threshold(s) 232. Threshold(s) 232 may include a threshold amount which may be used to determine whether to reschedule a subsequent medical procedure. The threshold amount may be an amount of time. Threshold(s) 232 may include a threshold time which may be used to determine whether to prompt a clinician to seek assistance. These thresholds are discussed further later in this disclosure.

Memory 202 may include scheduling information 234. For example, scheduling information 234 may include information relating to scheduled medical procedures, such as those medical procedures scheduled for a current day, week or other time period. Scheduling information 234 may include predicted durations, start times, and/or end times for subsequent medical procedures. In some examples, when processing circuitry 204 outputs a scheduling indication, the scheduling indication may be stored in scheduling information 234.

Memory 202 may also store one or more machine learning model(s) 222 and user interface 218. Machine learning model(s) 222 may be configured to, when executed by processing circuitry 204, to determine estimated time for procedure 226, determine that an event occurs in a current medical procedure, and/or revised estimated time for procedure 228.

For example, processing circuitry 204 executing machine learning model(s) 222 may determine estimated time for procedure 226 based on a type of procedure, clinicians scheduled to perform the procedure, image data 214 (which may include image data from a current medical procedure, as well as image data from one or more previous medical procedures), audio data 216, other data 224, video data 230, and/or the like.

In some examples, processing circuitry 204 may execute machine learning model(s) 222 to determine revised estimated time for procedure 228. For example, processing circuitry 204 executing machine learning model(s) 222 may monitor progress of the current medical procedure through image data 214, audio data 216, other data 224, and/or video data 230. Processing circuitry 204 executing machine learning model(s) 222 may determine an event has occurred during the current medical procedure based on one or more of image data 214, audio data 216, other data 224, and/or video data 230. For example, a medical instrument may appear in image data 214 that may indicate that a current diagnostic medical procedure has now become an interventional medical procedure. This may be an example event. Other example events are discussed further hereinafter. Based on determining the event has occurred, processing circuitry 204 executing machine learning model(s) 222 may determine revised estimated time for procedure 228. For example, machine learning model(s) 222 may have been trained on training data which may include the occurrence of that event or a similar event and may be able to determine revised estimated time for procedure 228 based on times associated with the occurrence of that event or a similar event in the training data.

In such examples, machine learning model(s) 222 may be trained using image data, audio data, video data, and/or other data of a plurality of medical procedures, durations associated with the plurality of medical procedures, durations between events and/or an end of the plurality of medical procedures, durations associated with clinicians and/or clinician roles in the plurality of medical procedures, annotations, and/or the like.

In some examples, machine learning model(s) 222 may be trained to recognize various medical instruments and/or devices in image data 214, in video data 230, and/or based on other data 224 (e.g., based on a scanned barcode). As such, processing circuitry 204 executing machine learning model(s) 222 may determine a state of the medical procedure, such as a phase of the medical procedure based on which medical instrument(s) and/or device(s) may be being used in the medical procedure.

In some examples, machine learning model(s) 222 may be trained to recognize spoken words in audio data 216. As such, processing circuitry 204 executing machine learning model(s) 222 may determine a state of the medical procedure, such as a phase of the medical procedure based on which words spoken and captured by one or more microphone(s) of additional equipment 170 during the medical procedure.

In some examples, processing circuitry 204 executing machine learning model(s) 222 may continuously monitor image data 214, audio data 216, other data 224, and/or video data 230 for indications of any events which may impact a duration or end time of the current medical procedure so that processing circuitry 204 may determine revised estimated time for procedure 228 upon occurrence of such an event. In this manner, processing circuitry 204 may, for example, via network interface 208, output a scheduling indication. For example, the scheduling indication may include a notification to a patient scheduled for a upcoming or subsequent medical procedure and/or scheduling staff of a likelihood of a need to reschedule the upcoming or subsequent medical procedure and/or may automatically reschedule the upcoming or subsequent medical procedure when the current medical procedure is estimated to impact the schedule (e.g., scheduled time, location, etc.) of the upcoming or subsequent medical procedure. For example, in some cases, a 1 minute difference between estimated time for procedure 226 and revised estimated time for procedure 228 may not impact scheduling and may not trigger processing circuitry to output the scheduling indication. In some examples, processing circuitry 204 may determine whether revised estimated time for procedure 228 varies (e.g., differs) more than a threshold amount (of time) of threshold(s) 232 from estimated time for procedure 226 before outputting a scheduling indication.

Additionally, because more than one phase of a medical procedure may take longer than predicted or less time than predicted, or phases may be added to or subtracted from a medical procedure more than once, it may be desirable to selectively output scheduling indications. As such, in some examples, prior to outputting a scheduling indication, processing circuitry 204 may determine whether the revised predicted duration or the revised predicted end time varies by more than a threshold amount from the predicted duration or the predicted end time. For example, if the revised predicted duration or the revised predicted end time does vary by more than a threshold amount from the predicted duration or the predicted end time, this may be considered a significant scheduling impact and processing circuitry 204 may output the scheduling indication. If the revised predicted duration or the revised predicted end time does not vary by more than a threshold amount from the predicted duration or the predicted end time, in some examples, processing circuitry 204 may not output the scheduling indication. In this manner, a scheduling indication may not be output for every time a new revised estimated time for procedure is determined, which may reduce the number of notifications sent to the patient and/or scheduling staff when scheduling impact is minimal. In some examples, this threshold amount is programmable by a user. The threshold amount may be a fixed amount of time or an amount of time that may be based on other factors. It may be desirable that scheduling indications only be output for scheduling inputs that would cause a subsequent medical procedure to be pushed from the current date to another date. As such, the threshold amount may take into account scheduling information 234 relating to subsequent medical procedures, the current time of day, or the like.

User input data of other data 224, such as the type of medical procedure, identification of clinicians involved in the current medical procedure, and/or roles of each such clinician, may be useful to processing circuitry 204 in determining estimated time for procedure 226, as different types of medical procedures may take different amounts of time to complete, and different clinicians may take different amounts of time to perform the same type of procedure. Prior to a first medical procedure, users (e.g., clinicians) may fill out simple profiles detailing their name and/or badge number and the roles they may typically fill in the Cath lab or operating room. In some examples, a user may also enter a room in which computing device 150 resides. Such information may be entered into computing device 150 and/or server 160 via an input device, user interface, or the like.

With regards to a particular medical procedure, a user may enter or select the type of medical procedure, the profiles (or other identification) of the clinicians involved in the medical procedure and, if appropriate, the role of each such clinician, e.g., via input device(s) 210, user interface 218, display 206, network interface 208, or the like. Processing circuitry 204 may store such information in other data 224 and use such information to determine estimated time for procedure 226. In some examples, rather than determine estimated time for procedure 226 based on the information referred to above, processing circuitry 204 may determine estimated time for procedure 226 based on an estimate of the duration or end time of the procedure entered by a user.

Over time, processing circuitry 204 may determine trends for specific clinician teams, such as physicians or combinations of physicians and staff, and their impact on medical procedure time. For example, if a particular clinician typically needs more time to complete one type of medical procedure, processing circuitry may take this into account when determining estimated time for procedure 226 for subsequent medical procedures involving the particular clinician, for example, in the same role, performing the same type of procedure.

In some examples, processing circuitry 204 may provide information to scheduling staff in advance including an estimated time for procedure 226 for a plurality of medical procedures to assist the scheduling staff in scheduling the plurality of medical procedures. In some examples, processing circuitry 204 may automatically schedule the plurality of medical procedures.

During a medical procedure, if processing circuitry 204 detects something that may shorten or lengthen the medical procedure (e.g., for example, based on image data 214, audio data 216, video data 230, and/or other data 224), processing circuitry 204 may output a scheduling indication to push that information to scheduling staff and/or may automatically reschedule patients and/or resources. For scheduling staff, this advanced knowledge may allow for the better management of the pool of patients and hospital resources. For example, the advanced knowledge may allow a scheduling staff to notify a patient that their medical procedure will be bumped to the following day, shift, or week, reducing or avoiding time spent by the patient in a waiting room the day on which the procedure was originally scheduled.

With a coronary medical procedure, processing circuitry 204 executing machine learning model(s) 222 may detect a switch from a diagnostic medical procedure to an interventional medical procedure, for example, by detecting the introduction of a medical instrument into the vasculature of the patient in image data 214. For example, processing circuitry 204 executing machine learning model(s) 222 may identify a change from a diagnostic catheter to a guide catheter and the introduction of a crossing balloon in fluoroscopy image data of image data 214. Processing circuitry 204 executing machine learning model(s) 222 may then determine that such action is likely to extend the medical procedure time by a particular amount or range of time (e.g., X-Y minutes).

With an RDN procedure, processing circuitry 204 executing machine learning model(s) 222 may determine, based on an initial patient scan (e.g., based on image data 214) that a generator will need to provide 14 energy deliveries per protocol. The example patient may have a tortuous anatomy and sleep apnea. Processing circuitry 204 executing machine learning model(s) 222 may determine that previous medical procedures similar to the RDN procedure have taken between X and Y minutes to complete.

With a chronic total occlusion (CTO) medical procedure, the clinician may start with an antegrade approach, but convert to retrograde approach, which would require more time.

Processing circuitry 204 executing machine learning model(s) 222 may recognize in image data 214 the change to the retrograde approach and may determine that previous medical procedures similar to the current CTO procedure have taken between X and Y minutes to complete once the change to the retrograde approach has been made.

In some examples, where there are multiple options available for treatment, such as with a CTO, (e.g., antegrade and retrograde approaches), processing circuitry 204 may include enough time for both approaches when determining estimated time for procedure 226. In this manner, any determined revised estimated time for procedure 228 is very likely to be of a shorter duration than estimated time for procedure 226, which may make it less likely that a subsequent medical procedure may be pushed back in time.

In some examples, system 100 may provide a “check-in” feature. In the example where computing device 150 may notify scheduling staff that there is a delay in the current medical procedure, processing circuitry 204 may, for example, upon request from a user, such as scheduling staff, determine an estimated time until a next phase of the current procedure is completed. For example, a delay in the current medical procedure may be caused by a diagnostic procedure becoming an interventional procedure. Such a delay may be a relatively large delay or a relatively small delay. The extent of such a delay may be more apparent or determinable once intravascular imaging (e.g., IVUS) imaging is taken and analyzed. As such, processing circuitry 204 may control display 206, display device 110, our output device(s) 212, to output a message such as “Intravascular imaging is typically taken in the next 10 minutes, please check back.” Such a feature may reduce the amount of interactions the scheduling staff may have with the clinicians performing the current medical procedure, thereby increasing the time the clinicians performing the current medical procedure may spend focused on the current medical procedure itself. In some examples, the scheduling staff may request that processing circuitry provide updates on the progress of the current medical procedure. In some examples, such updates may be provided based on progress through each of a number of phases of the current medical procedure.

Because processing circuitry 204 may have knowledge of clinicians and their roles associated with a medical procedure, as well as the time such clinicians may take to complete each medical procedure, over time, processing circuitry 204 may improve staffing by suggesting to scheduling staff clinicians that perform relatively well with each other. Processing circuitry 204 may also identify clinicians that are most efficient at specific procedures and recommend such clinicians for those specific procedures. Processing circuitry 204 may also draw associations between specific equipment and/or technology used and any associated reduced medical procedure time. Processing circuitry 204 may therefore, highlight or propose treatment strategies associated with reduced medical procedure time to, e.g., clinicians.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated (e.g., via one or more microphones of additional equipment 170), enabling display 206 to serve as both an input and output device. Alternatively, a keyboard (not shown), mouse (not shown), or other data input devices (e.g., input device(s) 210) may be employed.

Network interface 208 may be adapted to connect to a network such as a local area network (LAN) that includes a wired network or a wireless network, a wide area network (WAN), a wireless mobile network, a Bluetooth network, or the Internet. For example, computing device 150 may obtain image data 214 from imager 140 during a medical procedure. Computing device 150 may receive updates to its software, for example, application(s) 217, via network interface 208. Computing device 150 may also display notifications on display 206 that a software update is available.

Input device(s) 210 may include any device that enables a user to interact with computing device 150, such as, for example, a mouse, keyboard, foot pedal, touch screen, augmented-reality input device receiving inputs such as hand gestures or body movements, or voice interface.

Output device(s) 212 may include any connectivity port or bus, such as, for example, parallel ports, serial ports, universal serial busses (USB), or any other similar connectivity port known to those skilled in the art.

Application(s) 217 may be one or more software programs stored in memory 202 and executed by processing circuitry 204 of computing device 150. Processing circuitry 204 may execute user interface 218, which may display image data 214, other data 224, video data 230, estimated time for procedure 226, and/or revised estimated time for procedure 228 on display 206 and/or display device 110.

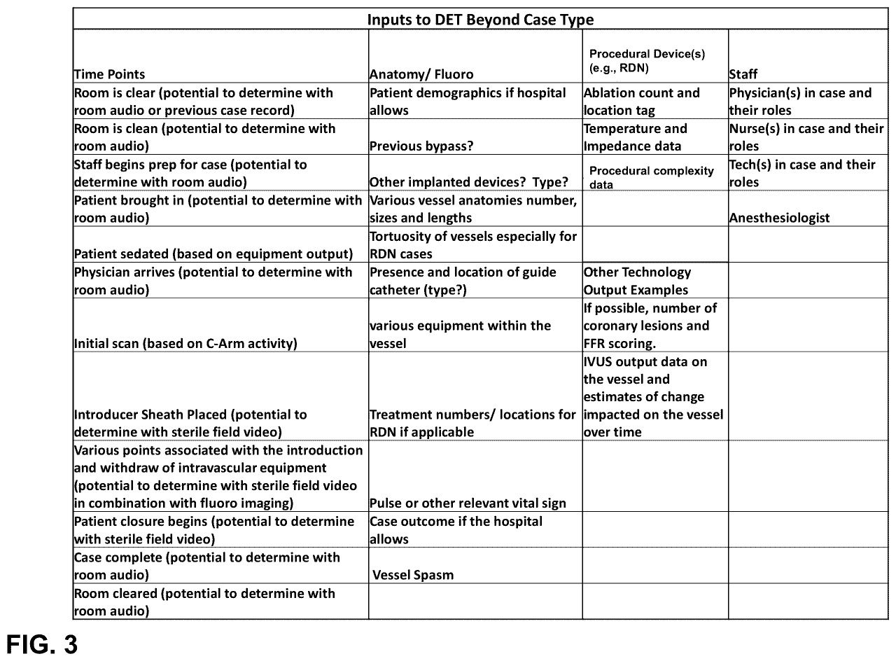

FIG. 3 is a tabular diagram illustrating example inputs that may be used to determine a duration or an end time and/or a revised duration or a revised end time of a medical procedure according to one or more aspects of this disclosure. In some examples, one or more of such inputs may be used by processing circuitry 204 to determine estimated time for procedure 226 and/or revised estimated time for procedure 228. For example, processing circuitry 204 may determine estimated time for procedure 226 based on the type of medical procedure to be performed (or being performed). Processing circuitry 204 may determine the type of medical procedure based on data associated with the medical procedure, such as image data 214, audio data 216, video data 230, and/or other data 224 (e.g., user input data and/or data from additional equipment 170). For example, the type of medical procedure may be included in other data 224 and have been input into computing device 150 by a user. In some examples, processing circuitry 204 may determine estimated time for procedure 226 based on information, in addition to, or in lieu of, the type of medical procedure.

In some examples, image data 214 may include patient demographic information, evidence of previous interventions (e.g., a bypass), the existence and/or type of existing implanted device(s), vessel information (e.g., vessel morphology), presence of medical instruments and/or medical equipment (e.g., guide catheters), treatment quantities and/or locations (e.g., for RDN treatments), vital signs, case outcome, vessel spasm, and/or the like. In some examples, processing circuitry 204 executing machine learning model(s) 222 may utilize such information when determining estimated time for procedure 226, and/or revised estimated time for procedure 228.

In some examples, other data 224 may include data from a procedural device, such as an RDN device, a laparoscopic tool, a surgical tool, an anesthesia delivery device, FFR device, a barcode scanner, or the like. For example, other data 224 may include ablation quantity and locations, temperature and impedance data, measures or indications of procedural complexity, lesion quantity and/or morphology, FFR score(s), estimates of change impact on a vessel over time, or the like. In some examples, other data 224 may include user input data, such as clinician identification of clinicians involved in the medical procedure, clinician roles in the medical procedure, or the like. In some examples, processing circuitry 204 executing machine learning model(s) 222 may utilize such information when determining estimated time for procedure 226, and/or revised estimated time for procedure 228. While an RDN device is specifically mentioned in FIG. 3, the RDN device is set forth as an example. Other data 224 may include data from any of or any combination of devices of additional equipment 170 discussed herein.

For example, audio data 216, video data 230, and/or other data 224 (e.g., user input data and/or data from additional equipment 170) may be used by processing circuitry 204 executing machine learning model(s) 222 to determine various time points or phases associated with estimated time for procedure 226 and/or revised estimated time for procedure 228. For example, processing circuitry 204 executing machine learning model(s) 222 may determine that a room (e.g., operating room, Cath lab, or the like) is clear, that the room is clean, that staff has begun preparing for the medical procedure, that the patient is present in the room, that the patient is sedated, that the clinician is present in the room, that an initial scan by imager 140 has begun, that medical instruments and/or medical devices are introduced to and or withdrawn from the patient, that patient closure begins, the medical procedure ends, and/or that the room has been cleared. One or more of these time points may be considered to mark the beginning or end of a phase of the medical procedure.

In some examples, processing circuitry 204 may support the use of custom tags for events. For example, a user may designate an event as being a milestone to be monitored. The system may then monitor for the events, such as through image data 214, audio data 216, video data 230, and/or other data 224. For example, an event may include placement of a medical instrument or equipment, such as guide catheter placement or an RDN device, energy delivery, or the like. In some examples, processing circuitry 204 may flag anatomy and/or patient reactions (e.g., vessel spasm) as events that may be indicative of a predicted change in duration of the current medical procedure. For example, processing circuitry 204 may, for an RDN procedure, flag vessels that meet ablation criteria, tortuosity scoring, etc., as being an event.

Machine learning model(s) 222 may be trained to determine what phases are included in a particular type of medical procedure and to predict a time duration associated with each phase of the medical procedure. In this manner, processing circuitry 204 executing machine learning model(s) 222 may predict estimated time for procedure 226. While monitoring data associated with the medical procedure, such as image data 214, audio data 216, video data 230, and/or other data 224, processing circuitry 204 executing machine learning model(s) 222 may determine an event occurs in the medical procedure. This event may be indicative of the end of a phase or the beginning of a phase of the medical procedure or the beginning of a phase not previously associated with the medical procedure (e.g., the introduction of an invasive medical instrument to the patient during a diagnostic medical procedure). Machine learning model(s) 222 may be trained to determine revised estimated time for procedure 228 based on such events. For example, if a new phase is added to the medical procedure, processing circuitry 204 executing machine learning model(s) 222 may reclassify the medical procedure adding that new phase and any other associated phases expected by machine learning model(s) 222 to now be part of the medical procedure and revise the original estimated time for procedure 226 based on expected time durations for the new phase(s) of the medical procedure. Processing circuitry 204 may store this revision as revised estimated time for procedure 228.

To determine estimated time for procedure 226, and/or revised estimated time for procedure 228, machine learning model(s) 222 may have been trained to associate various phases with a given type of medical procedure and a time duration associated with each phase. In this manner, processing circuitry 204 executing machine learning mode(s) 222 may determine estimated time for procedure 226. When processing circuitry 204 executing machine learning model(s) 222 determines an event that may impact estimated time for procedure 226 (e.g., a new phase, an early or late ending to a phase, or the like), processing circuitry 204 executing machine learning model(s) 222 may determine revised estimated time for procedure 228 based on the occurrence of the event.

For example, once trained, processing circuitry 204 executing machine learning model(s) 222 may identify medical procedures that are likely to extend beyond estimated time for procedure 226, for example, based on previous similar patients (procedure type, age, gender, BMI, etc.). Processing circuitry 204 may also include other factors, such as in which room the medical procedure is being performed, the identity and/or roll of the clinicians performing the procedure, or the like. Processing circuitry 204 may determine an expected duration of the medical procedure, which in some examples, may include a range of durations. Scheduling staff may use such an estimate to assist with front end scheduling. During the medical procedure, processing circuitry 204 may provide live updates of progress of the medical procedure (e.g., of phases occurring) back to the scheduling staff to assist with rescheduling clinician, equipment, and/or patient schedules. For example, if a complex renal anatomy is captured on fluoroscopy in an RDN medical procedure, or a clinician introduces therapeutic devices (ex. crossing balloons) in a scheduled diagnostic medical procedure (e.g., a medical procedure not expected to include therapy), processing circuitry 204 may determine an expected impact of such unanticipated events and notify hospital staff as to schedule impacts. These live updates may be used to give as much advanced notice as possible to the staff and/or patients involved with subsequent medical procedures or with scheduling thereby allowing for greater resource flexibility.

Different phases of a medical procedure and predicted times associated therewith, may be used to allow for scheduling resources in a staggered manner for the medical procedure. For example, it may be desirable to administer anesthesia about X minutes after the medical procedure starts so as to avoid negatively impacting the medical procedure duration. This information may also help with staggering the timing of resources after the medical procedure, such as patient room availability and nursing staff.

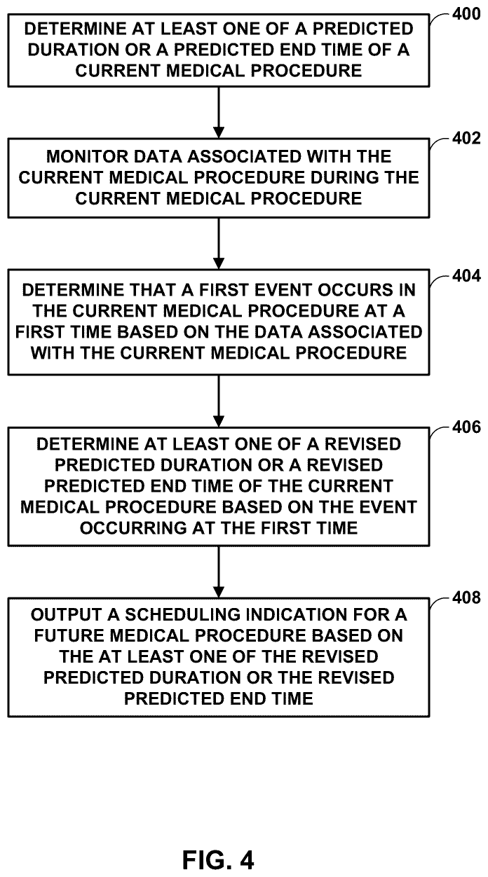

FIG. 4 is a flow diagram illustrating example medical procedure rescheduling techniques according to one or more aspects of this disclosure. The techniques of FIG. 4 are described below with respect to processing circuitry 204, but such techniques may be performed by any of, or any combination of, processing circuitry of devices depicted in FIG. 1 or capable of performing such techniques.

Processing circuitry 204 may determine at least one of a predicted duration or a predicted end time of a current medical procedure (400). For example, processing circuitry 204 may execute machine learning model(s) 222 on input data from image data 214, audio data 216, other data 224, and/or video data 230 to determine estimated time for procedure 226. In some examples, processing circuitry 204 may determine estimated time for procedure 226 without executing machine learning model(s) 222. For example, a clinician may enter other data 224 which may include a predicted duration and/or a predicted end time of the current medical procedure. In such a case, processing circuitry 204 may simply use the predicted duration and/or a predicted end time as estimated time for procedure 226.

Processing circuitry 204 may monitor data associated with the current medical procedure during the current medical procedure (402). For example, processing circuitry 204 executing machine learning model(s) 222 may monitor image data 214, audio data 216, video data 230, and/or other data 224 during the current medical procedure.

Processing circuitry 204 may determine that an event occurs in the current medical procedure at a first time based on the data associated with the current medical procedure (404). For example, processing circuitry 204 executing machine learning model(s) 222 may determine the event occurs based on image data 214, audio data 216, video data 230, and/or other data 224 collected during the medical procedure.

Processing circuitry 204 may determine at least one of a revised predicted duration or a revised predicted end time of the current medical procedure based on the event occurring at the first time (406). For example, processing circuitry 204 executing machine learning model(s) 222 may determine revised estimated time for procedure 228 based on the determination of the event. For example, the event may be indicative of an additional phase or phases being added to the current medical procedure which may lengthen the actual duration of the current medical procedure, thereby impacting a schedule of a subsequent medical procedure.

Processing circuitry 204 may output a scheduling indication for a subsequent medical procedure based on the at least one of the revised duration or the revised end time (408). For example, processing circuitry 204 may output a scheduling indication to a patient, scheduling staff, a scheduling algorithm, or the like. The scheduling indication may include an indication of revised estimated time for procedure 228, a scheduling impact to the subsequent medical procedure, or the like. In some examples, the scheduling indication may include an automatic rescheduling of the subsequent medical procedure. For example, the scheduling indication may cause the rescheduling of elements associated with the medical procedure, such as time, date, location, equipment, clinician(s) involved in the subsequent medical procedure, and may include or cause a notification to the patient of any of the rescheduled elements.

When automatically determining whether to automatically reschedule the subsequent medical procedure, in some examples, processing circuitry 204 may determine a risk to the health of the patient associated with rescheduling the subsequent procedure. If such a risk is relatively high (e.g., meets a threshold), processing circuitry 204 may not automatically reschedule the subsequent procedure (e.g., allowing the subsequent procedure to proceed on a delayed basis, but in a same facility on a same date). If such a risk is not relatively high (e.g., does not meet the threshold), processing circuitry 204 may reschedule the subsequent procedure.

When automatically rescheduling the subsequent medical procedure, in some examples, processing circuitry 204 may determine a risk associated with each of one or more medical procedures scheduled or to be scheduled on a given day, the risk being a risk that a respective medical procedure of the one or more medical procedures takes a longer time to complete than is scheduled. For example, if such risks are relatively high (e.g., meet a threshold), such that the subsequent medical procedure may have to be rescheduled again, processing circuitry 204 may forgo rescheduling the subsequent medical procedure for that given day.

In some examples, the event lengthens a duration of the current medical procedure. In some examples, the event includes at least one of an end of a phase of the medical event, a beginning of a phase of the medical event, a patient reaction, or an identification of new medical instrument being used.

In some examples, the data associated with the current medical procedure comprises at least one of image data 214, audio data 216, video data 230, user input data (of other data 224), fractional flow reserve data (of other data 224), or renal artery denervation data (of other data 224).

In some examples, the current medical procedure includes a plurality of phases. In some examples, as part of predicting the at least one of a revised duration or end time of the medical procedure, processing circuitry 204 is configured to at least one of add a new phase from the current medical procedure or remove an existing phase from the current medical procedure.

In some examples, the predicted duration or the predicted end time of the current medical procedure is based at least in part on at least one of a type of the current medical procedure, clinician identification, or clinician roles. For example, estimated time for procedure 226 may be based the type of the current medical procedure, identities of clinicians involved in the current medical procedure, and/or clinician roles assigned to clinicians involved in the current medical procedure.

In some examples, processing circuitry 204 is configured to determine that the event has not occurred by a threshold time, for example, of threshold(s) 232. Based on the event not occurring by the threshold time, processing circuitry 204 may prompt a clinician to seek assistance. For example, processing circuitry 204 may output to display 206 and/or display device 110 a message asking if the clinician would like to contact a manufacturer's representative, another clinician, another specialty group (e.g., a heart team), or the like.

In some examples, processing circuitry 204 is configured to determine that the event has not occurred after use of a threshold number of therapy devices being utilized during the current medical procedure. The threshold number of devices may be stored in threshold(s) 232. Based on the event not occurring after the use of the threshold number of therapy devices, processing circuitry 204 may prompt a clinician to seek assistance. For example, processing circuitry 204 may output to display 206 and/or display device 110 a message asking if the clinician would like to contact a manufacturer's representative, another clinician, another specialty group (e.g., a heart team), or the like.

In some examples, as part of at least one of determining the at least one of the predicted duration or the end time, or determining the at least one of a revised duration or revised end time of the medical procedure, processing circuitry 204 may executed machine learning model(s) 222. Machine learning model(s) 222 may be trained on at least one of image data, audio data, video data, user input data, renal artery denervation device data, or fractional flow reserve data.

In some examples, as part of outputting the scheduling indication, processing circuitry 204 is configured to at least one of automatically reschedule the subsequent medical appointment or send a notification to administration staff responsible for scheduling. In some examples, as part of automatically rescheduling the subsequent medical appointment, processing circuitry 204 may reschedule at least one of staff, hospital equipment, rooms, or a patient. In some examples, as part of outputting the scheduling revision, processing circuitry 204 is configured to send a notification to an electronic device (e.g., patient device 180 of FIG. 1) associated with a patient, wherein the notification comprises at least one of a new time, a new date, or a new medical facility for the subsequent medical procedure.

In some examples, processing circuitry 204 is configured to send the notification to administration staff responsible for scheduling, and processing circuitry 204 is further configured to receive a request from a user to determine an estimated time until a next phase of the current procedure is completed. In such examples, processing circuitry 204 is configured to determine the estimated time until the next phase of the current procedure is completed and output an indication of the estimated time until the next phase of the current procedure is completed.

In some examples, processing circuitry 204 is further configured to determine that at least one phase of the medical procedure is completed prior to an expected end time of the at least one phase and identify one or more techniques used during the at least one phase based on imaging data. In some examples, processing circuitry 204 is further configured to determine that the medical procedure is completed at least one of in a shorter duration then the predicted duration or prior to the predicted end time of the medical procedure and identify a team of clinicians associated with the medical procedure as an efficient team.

In some examples, processing circuitry 204 is further configured to determine that the revised predicted duration or the revised predicted end time varies by more than a threshold amount from the predicted duration or the predicted end time. In some examples, outputting the scheduling indication is further based on the revised predicted duration or the revised predicted end time varying by more than the threshold amount from the predicted duration or the predicted end time. In some examples, the threshold amount is programmable.

In some examples, the event is a first event, the revised predicted duration is a first revised predicted duration, the revised predicted end time is a first revised predicted end time, and the scheduling indication is a first scheduling indication. In some examples, processing circuitry 204 is further configured to determine that a second event occurs in the current medical procedure based on the data associated with the current medical procedure. In such examples, processing circuitry 204 is further configured to determine at least one of a second revised predicted duration or a second revised predicted end time of the current medical procedure based on the second event occurring and output a second scheduling indication for a subsequent medical procedure based on the at least one of the revised predicted duration or the revised predicted end time.

In some examples, processing circuitry 204 is configured to determine at least one of a respective predicted duration or a respective predicted end time for each of a plurality of current medical procedures. In such examples, processing circuitry 204 is configured to monitor respective data associated with each of the plurality of current medical procedures during each of the plurality of current medical procedures. In such examples, processing circuitry 204 is configured to determine that a respective event occurs in at least two of the plurality of current medical procedures based on the respective data associated with each of the plurality of current medical procedures. In such examples, processing circuitry 204 is configured to determine at least one of a respective revised predicted duration or a respective revised predicted end time of each of the at least two of the plurality of current medical procedures based on the respective event occurring. In such examples, processing circuitry 204 is configured to output a respective scheduling indication for each of a plurality of subsequent medical procedures based on at least one of the at least one respective revised predicted duration or revised predicted end time. For example, the techniques of this disclosure may be used to monitor each medical procedure being performed in a given medical facility and perform rescheduling of medical procedures based on events occurring within current medical procedures so as to reduce or minimize waiting room times for patients of subsequent medical procedures.

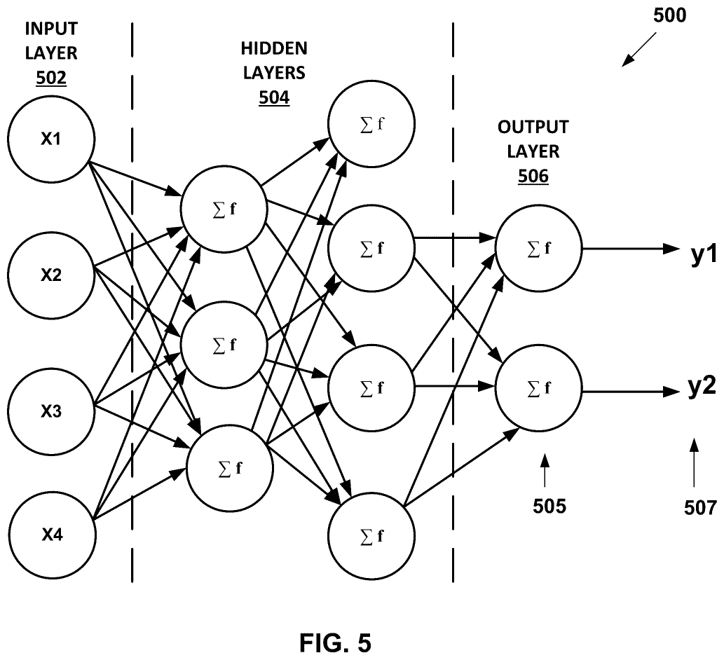

FIG. 5 is a conceptual diagram illustrating an example machine learning model according to one or more aspects of this disclosure. Machine learning model 500 may be an example of machine learning model(s) 222. Machine learning model 500 may be an example of a deep learning model, or deep learning algorithm, trained to determine an identification of an implanted medical device and/or to determine a recommended treatment strategy. One or more of computing device 150 and/or server 160 may train, store, and/or utilize machine learning model 500, but other devices of system 100 may apply inputs to machine learning model 500 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron. In some examples, machine learning model 500 may be a continuous learning model which may use input data to improve training over time.

As shown in the example of FIG. 5, machine learning model 500 may include three types of layers. These three types of layers include input layer 502, hidden layers 504, and output layer 506. Output layer 506 comprises the output from the transfer function 505 of output layer 506. Input layer 502 represents each of the input values X1 through X4 provided to machine learning model 500. In some examples, the input values may include any of the values input into the machine learning model, as described above. For example, the input values may include or be based on image data 214, audio data 216, video data 230, and/or other data 224, as described above. In addition, in some examples, input values of machine learning model 500 may include additional data, such as other data that may be collected by or stored in system 100.

Each of the input values for each node in the input layer 502 is provided to each node of a first layer of hidden layers 504. In the example of FIG. 5, hidden layers 504 include two layers, one layer having four nodes and the other layer having three nodes, but fewer or greater number of nodes may be used in other examples. Each input from input layer 502 is multiplied by a weight and then summed at each node of hidden layers 504. During training of machine learning model 500, the weights for each input are adjusted to establish the relationship between image data 214, audio data 216, video data 230, and/or other data 224, and estimated time for procedure 226 and/or revised estimated time for procedure 228. In some examples, one hidden layer may be incorporated into machine learning model 500, or three or more hidden layers may be incorporated into machine learning model 500, where each layer includes the same or different number of nodes.

The result of each node within hidden layers 504 is applied to the transfer function of output layer 506. The transfer function may be linear or non-linear, depending on the number of layers within machine learning model 500. Example non-linear transfer functions may be a sigmoid function or a rectifier function. The output 507 of the transfer function may be a classification that image data 214, audio data 216, video data 230, and/or other data 224 is indicative of a particular phase of a medical procedure and/or a duration to complete the medical procedure.

As shown in the example above, by applying machine learning model 500 to input data such as image data 214, audio data 216, video data 230, and/or other data 224, processing circuitry 204 is able to determine estimated time for procedure 226 and/or revised estimated time for procedure 228. This may improve the efficiency of the use of medical resources, the adoption of techniques to improve the time a medical procedure may take, the ability to reschedule patients of subsequent medical procedures earlier, and/or the like.

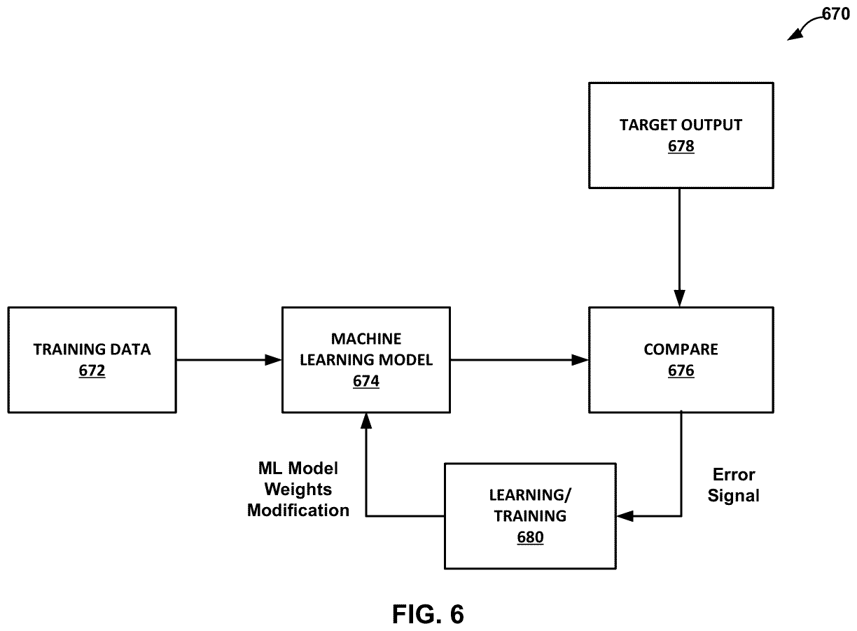

FIG. 6 is a conceptual diagram illustrating an example training process for a machine learning model according to one or more aspects of this disclosure. Process 670 may be used to train machine learning model(s) 222 or machine learning model 500. A machine learning model 674 (which may be an example of machine learning model 500 and/or machine learning model(s) 222) may be implemented using any number of models for supervised and/or reinforcement learning, such as but not limited to, an artificial neural network, a decision tree, naïve Bayes network, support vector machine, or k-nearest neighbor model, CNN, RNN, LSTM, ensemble network, to name only a few examples. In some examples, one or more of computing device 150 and/or server 160 initially trains machine learning model 674 based on a corpus of training data 672. Training data 672 may include, image data, audio data, video data, and/or other data (e.g., user input data, data from other equipment, etc.), of a plurality of past medical procedures performed on a plurality of patients having different patient conditions, durations associated with the plurality of medical procedures, durations between events and/or an end of the plurality of medical procedures, durations associated with clinicians and/or clinician roles in the plurality of medical procedures, annotations, other training data mentioned herein, and/or the like.

While training machine learning model 674, processing circuitry of system 100 may compare 676 a prediction or classification with a target output 678. Processing circuitry 204 may utilize an error signal from the comparison to train (learning/training 680) machine learning model 674. Processing circuitry 204 may generate machine learning model weights or other modifications which processing circuitry 204 may use to modify machine learning model 674. For examples, processing circuitry 204 may modify the weights of machine learning model 674 based on the learning/training 680. For example, one or more of computing device 150 and/or server 160, may, for each training instance in training data 672, modify, based on training data 672, the manner in which estimated time for procedure 226 and/or revised estimated time for procedure 228 is determined.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1. A medical system comprising: memory configured to store at least one of a predicted duration or predicted end time of a current medical procedure; and processing circuitry communicatively coupled to the memory, the processing circuitry being configured to: determine at least one of the predicted duration or the predicted end time; monitor data associated with the current medical procedure during the current medical procedure; determine that an event occurs in the current medical procedure at a first time based on the data associated with the current medical procedure; determine at least one of a revised predicted duration or a revised predicted end time of the current medical procedure based on the event occurring at the first time; and output a scheduling indication for a subsequent medical procedure based on the at least one of the revised predicted duration or the revised predicted end time.

Example 2. The medical system of example 1, wherein the event lengthens a duration of the current medical procedure.

Example 3. The medical system of example 1 or example 2, wherein the event comprises at least one of an end of a phase of the medical event, a beginning of a phase of the medical event, a patient reaction, or an identification of new medical instrument being used.

Example 4. The medical system of any of examples 1-3, wherein the data associated with the current medical procedure comprises at least one of image data, audio data, video data, user input data, fractional flow reserve data, or procedural device data.

Example 5. The medical system of any of examples 1-4, wherein the current medical procedure comprises a plurality of phases.

Example 6. The medical system of example 5, wherein as part of predicting the at least one of a revised duration or end time of the medical procedure, the processing circuitry is configured to at least one of add a new phase from the medical procedure or remove an existing phase from the medical procedure.

Example 7. The medical system of any examples 1-6, wherein the predicted duration or the predicted end time of the current medical procedure is based at least in part on at least one of a type of the current medical procedure, clinician identification, or clinician roles.

Example 8. The medical system of examples 1-7, wherein the processing circuitry is further configured to: determine that the event has not occurred by a threshold time; and based on the event not occurring by the threshold time, prompting a clinician to seek assistance.

Example 9. The medical system of any of examples 1-8, wherein as part of at least one of determining the at least one of the predicted duration or the end time, or determining the at least one of a revised duration or revised end time of the medical procedure, the processing circuitry is configured to execute a machine learning model, and wherein the machine learning model is trained on at least one of image data, audio data, video data, user input data, procedural device data, or fractional flow reserve data.

Example 10. The medical system of any of examples 1-9, wherein as part of outputting the scheduling indication, the processing circuitry is configured to at least one of automatically reschedule the subsequent medical appointment or send a notification to administration staff responsible for scheduling.

Example 11. The medical system of example 10, wherein as part of automatically rescheduling the subsequent medical appointment, the processing circuitry is configured to reschedule at least one of staff, hospital equipment, rooms, or a patient.

Example 12. The medical system of example 10 or example 11, wherein as part of outputting the scheduling revision, the processing circuitry is configured to send a notification to an electronic device associated with a patient, wherein the notification comprises at least one of a new time, a new date, or a new medical facility for the subsequent medical procedure.

Example 13. The medical system of any of examples 10-12, wherein the processing circuitry is configured to send the notification to administration staff responsible for scheduling, and wherein the processing circuitry is further configured to: receive a request from a user to determine an estimated time until a next phase of the current procedure is completed; determine the estimated time until the next phase of the current procedure is completed; and output an indication of the estimated time until the next phase of the current procedure is completed.

Example 14. The medical system of any of examples 1-13, wherein the processing circuitry is further configured to: determine that at least one phase of the medical procedure is completed prior to an expected end time of the at least one phase; and identify one or more techniques used during the at least one phase based on imaging data.

Example 15. The medical system of any of examples 1-14, wherein the processing circuitry is further configured to: determine that the medical procedure is completed at least one of in a shorter duration then the predicted duration or prior to the predicted end time of the medical procedure; and identify a team of clinicians associated with the medical procedure as an efficient team.

Example 16. The medical system of any of examples 1-15, wherein the processing circuitry is further configured to determine that the revised predicted duration or the revised predicted end time varies by more than a threshold amount from the predicted duration or the predicted end time, wherein the outputting the scheduling indication is further based on the revised predicted duration or the revised predicted end time varying by more than the threshold amount from the predicted duration or the predicted end time.

Example 17. The medical system of any of examples 1-16, wherein the event is a first event, the revised predicted duration is a first revised predicted duration, the revised predicted end time is a first revised predicted end time, and the scheduling indication is a first scheduling indication, and wherein the processing circuitry is further configured to: determine that a second event occurs in the current medical procedure based on the data associated with the current medical procedure; determine at least one of a second revised predicted duration or a second revised predicted end time of the current medical procedure based on the second event occurring; and output a second scheduling indication for a subsequent medical procedure based on the at least one of the revised predicted duration or the revised predicted end time.

Example 18. The medical system of any of examples 1-17, wherein the processing circuitry is further configured to: determine at least one of a respective predicted duration or a respective predicted end time for each of a plurality of current medical procedures; monitor respective data associated with each of the plurality of current medical procedures during each of the plurality of current medical procedures; determine that a respective event occurs in at least two of the plurality of current medical procedures based on the respective data associated with each of the plurality of current medical procedures; determine at least one of a respective revised predicted duration or a respective revised predicted end time of each of the at least two of the plurality of current medical procedures based on the respective event occurring; and output a respective scheduling indication for each of a plurality of subsequent medical procedures based on at least one of the at least one respective revised predicted duration or revised predicted end time.

Example 19. A method comprising: determining, by processing circuitry, at least one of a predicted duration or a predicted end time of a current medical procedure; monitoring, by the processing circuitry, data associated with the current medical procedure during the current medical procedure; determining, by the processing circuitry, that an event occurs in the current medical procedure at a first time based on the data associated with the current medical procedure; determining, by the processing circuitry, at least one of a revised predicted duration or a revised predicted end time of the current medical procedure based on the event occurring at the first time; and outputting, by the processing circuitry, a scheduling indication for a subsequent medical procedure based on the at least one of the revised predicted duration or the revised predicted end time.

Example 20. A non-transitory computer-readable storage medium storing instructions, which when executed cause processing circuitry to: determine at least one of a predicted duration or a predicted end time of a current medical procedure; monitor data associated with the current medical procedure during the current medical procedure; determine that an event occurs in the current medical procedure at a first time based on the data associated with the current medical procedure; determine at least one of a revised predicted duration or a revised predicted end time of the current medical procedure based on the event occurring at the first time; and output a scheduling indication for a subsequent medical procedure based on the at least one of the revised predicted duration or the revised predicted end time.

Various examples have been described. These and other examples are within the scope of the following claims.

What is claimed is:

1. A medical system comprising:
memory configured to store at least one of a predicted duration or predicted end time of a current medical procedure; and
processing circuitry communicatively coupled to the memory, the processing circuitry being configured to:
determine at least one of the predicted duration or the predicted end time;
monitor data associated with the current medical procedure during the current medical procedure;
determine that an event occurs in the current medical procedure at a first time based on the data associated with the current medical procedure;
determine at least one of a revised predicted duration or a revised predicted end time of the current medical procedure based on the event occurring at the first time; and
output a scheduling indication for a subsequent medical procedure based on the at least one of the revised predicted duration or the revised predicted end time.

2. The medical system of claim 1, wherein the event lengthens a duration of the current medical procedure.

3. The medical system of claim 1, wherein the event comprises at least one of an end of a phase of the medical event, a beginning of a phase of the medical event, a patient reaction, or an identification of new medical instrument being used.

4. The medical system of claim 1, wherein the data associated with the current medical procedure comprises at least one of image data, audio data, video data, user input data, fractional flow reserve data, or procedural device data.

5. The medical system of claim 1, wherein the current medical procedure comprises a plurality of phases.

6. The medical system of claim 5, wherein as part of predicting the at least one of a revised duration or end time of the medical procedure, the processing circuitry is configured to at least one of add a new phase from the medical procedure or remove an existing phase from the medical procedure.

7. The medical system of claim 1, wherein the predicted duration or the predicted end time of the current medical procedure is based at least in part on at least one of a type of the current medical procedure, clinician identification, or clinician roles.

8. The medical system of claim 1, wherein the processing circuitry is further configured to:
determine that the event has not occurred by a threshold time; and
based on the event not occurring by the threshold time, prompting a clinician to seek assistance.

9. The medical system of claim 1, wherein as part of at least one of determining the at least one of the predicted duration or the end time, or determining the at least one of a revised duration or revised end time of the medical procedure, the processing circuitry is configured to execute a machine learning model, and wherein the machine learning model is trained on at least one of image data, audio data, video data, user input data, procedural device data, or fractional flow reserve data.

10. The medical system of claim 1, wherein as part of outputting the scheduling indication, the processing circuitry is configured to at least one of automatically reschedule the subsequent medical appointment or send a notification to administration staff responsible for scheduling.

11. The medical system of claim 10, wherein as part of automatically rescheduling the subsequent medical appointment, the processing circuitry is configured to reschedule at least one of staff, hospital equipment, rooms, or a patient.

12. The medical system of claim 10, wherein as part of outputting the scheduling revision, the processing circuitry is configured to send a notification to an electronic device associated with a patient, wherein the notification comprises at least one of a new time, a new date, or a new medical facility for the subsequent medical procedure.

13. The medical system of claim 10, wherein the processing circuitry is configured to send the notification to administration staff responsible for scheduling, and wherein the processing circuitry is further configured to:
receive a request from a user to determine an estimated time until a next phase of the current procedure is completed;
determine the estimated time until the next phase of the current procedure is completed; and
output an indication of the estimated time until the next phase of the current procedure is completed.

14. The medical system of claim 1, wherein the processing circuitry is further configured to:
determine that at least one phase of the medical procedure is completed prior to an expected end time of the at least one phase; and
identify one or more techniques used during the at least one phase based on imaging data.

15. The medical system of claim 1, wherein the processing circuitry is further configured to:
determine that the medical procedure is completed at least one of in a shorter duration then the predicted duration or prior to the predicted end time of the medical procedure; and
identify a team of clinicians associated with the medical procedure as an efficient team.

16. The medical system of claim 1, wherein the processing circuitry is further configured to determine that the revised predicted duration or the revised predicted end time varies by more than a threshold amount from the predicted duration or the predicted end time, wherein the outputting the scheduling indication is further based on the revised predicted duration or the revised predicted end time varying by more than the threshold amount from the predicted duration or the predicted end time.

17. The medical system of claim 1, wherein the event is a first event, the revised predicted duration is a first revised predicted duration, the revised predicted end time is a first revised predicted end time, and the scheduling indication is a first scheduling indication, and wherein the processing circuitry is further configured to:
determine that a second event occurs in the current medical procedure based on the data associated with the current medical procedure;
determine at least one of a second revised predicted duration or a second revised predicted end time of the current medical procedure based on the second event occurring; and
output a second scheduling indication for a subsequent medical procedure based on the at least one of the revised predicted duration or the revised predicted end time.

18. The medical system of claim 1, wherein the processing circuitry is further configured to:
determine at least one of a respective predicted duration or a respective predicted end time for each of a plurality of current medical procedures;
monitor respective data associated with each of the plurality of current medical procedures during each of the plurality of current medical procedures;
determine that a respective event occurs in at least two of the plurality of current medical procedures based on the respective data associated with each of the plurality of current medical procedures;
determine at least one of a respective revised predicted duration or a respective revised predicted end time of each of the at least two of the plurality of current medical procedures based on the respective event occurring; and
output a respective scheduling indication for each of a plurality of subsequent medical procedures based on at least one of the at least one respective revised predicted duration or revised predicted end time.

19. A method comprising:
determining, by processing circuitry, at least one of a predicted duration or a predicted end time of a current medical procedure;
monitoring, by the processing circuitry, data associated with the current medical procedure during the current medical procedure;
determining, by the processing circuitry, that an event occurs in the current medical procedure at a first time based on the data associated with the current medical procedure;
determining, by the processing circuitry, at least one of a revised predicted duration or a revised predicted end time of the current medical procedure based on the event occurring at the first time; and
outputting, by the processing circuitry, a scheduling indication for a subsequent medical procedure based on the at least one of the revised predicted duration or the revised predicted end time.

20. A non-transitory computer-readable storage medium storing instructions, which when executed cause processing circuitry to:
determine at least one of a predicted duration or a predicted end time of a current medical procedure;
monitor data associated with the current medical procedure during the current medical procedure;
determine that an event occurs in the current medical procedure at a first time based on the data associated with the current medical procedure;
determine at least one of a revised predicted duration or a revised predicted end time of the current medical procedure based on the event occurring at the first time; and
output a scheduling indication for a subsequent medical procedure based on the at least one of the revised predicted duration or the revised predicted end time.
